(19) United States
(12) Patent Application Publication
(10) Pub. No.: US 2016/0214106 A1
(43) Pub. Date: Jul. 28, 2016

(54) CAPILLARY DIAGNOSTIC DEVICE AND METHOD OF MAKING AND METHOD OF USE THEREOF

(71) Applicant: Materials and Machines Corporation of America, Lincoln, NE (US)

(72) Inventors: Abraham Oommen, Lincoln, NE (US); Matthew Greenleaf, Lincoln, NE (US)

(73) Assignee: Materials and Machines Corporation of America, Lincoln, NE (US)

(21) Appl. No.: 14/606,896

(22) Filed: Jan. 27, 2015

(51) Int. Cl. B01L 3/00; G01N 33/53

(52) U.S. Cl. CPC B01L 3/502707; G01N 33/5304; B01L 2300/0838; B01L 2300/06; B01L 2300/048; B01L 2200/12; B01L 2200/10; B01L 2300/163; B01L 2400/0406; B01L 2400/049

(57) ABSTRACT

A capillary diagnostic device comprising a cylindrical tube with one or more rings having one or more probes, the one or more rings disposed on the interior surface of the cylindrical tube. The invention also includes a method of making the capillary diagnostic device and method of use thereof for diagnostic testing.

### BACKGROUND

Diagnostic devices employ a variety of methods for detection of various analytes from biologic samples. Such devices are useful in diagnosing conditions, which allow for subsequent treatment or counseling of the subject.

### SUMMARY

Accordingly, an embodiment of the present disclosure includes a product and method for using a capillary diagnostic device. The present invention provides a unique device in which a probe (also known as an assay) is bound to the inside of a capillary in a localized, defined area. Constraining the probe to a localized, defined area improves detection because the probe is able to constrain an analyte to the same area.

It is to be understood that both the foregoing general description and the following detailed description are exemplary and explanatory only and are not necessarily restrictive of the disclosure as claimed. The accompanying drawings, which are incorporated in and constitute a part of the specification, illustrate embodiments of the disclosure and together with the general description, serve to explain the principles of the disclosure.

### BRIEF DESCRIPTION OF THE DRAWINGS

The numerous advantages of the present disclosure may be better understood by those skilled in the art by reference to the accompanying figures in which:

FIG. 1 is a cross sectional view of a cylindrical tube in accordance with an exemplary embodiment of the present disclosure.

FIG. 2 is a cross sectional view of a capillary detection device with one ring in accordance with an exemplary embodiment of the present disclosure.

FIG. 3 is a cross sectional view of a capillary detection device with a plurality of rings in accordance with an exemplary embodiment of the present disclosure.

FIG. 4 is a flow chart illustrating a method for production of the capillary detection device.

FIG. 5 is a flow chart illustrating a method for using the capillary detection device to detect an analyte of interest.

### DETAILED DESCRIPTION

Reference will now be made in detail to the embodiments of the present disclosure, examples of which are illustrated in the accompanying drawings. In this present disclosure, the terms “cylinder,” “cylindrical,” and “concentric” are not meant to limit cross-sectional shaping of the height and width for tubes embodied by the present disclosure. The term “cylinder” may also mean a tube with a triangle, square, pentagon, hexagon, heptagon, octagon or any other two-dimensional shape from the view of a height-wise and width-wise cross-section. The term “cylindrical” may also mean square-shaped, pentagonal-shaped, hexagonal-shaped, heptagonal-shaped, octagonal-shaped, or any other two-dimensional view of a tube's height-wise and width-wise cross-section. The term “concentric” means of or denoting a shape that shares the same center as another shape, the larger of the two shapes completely surrounding the smaller of the two shapes. The term “diameter” means a straight line passing from side to side through the center of a two-dimensional height-wise and width-wise cross-section of the cylinder.

Described herein is a capillary diagnostic device with a cylindrical tube, preferably made of glass or plastic, having one or more rings with one or more probes, the one or more rings disposed on (e.g., seated within) the interior surface of the cylindrical tube. Also described herein is the method of making the capillary diagnostic device, and a method for use thereof.

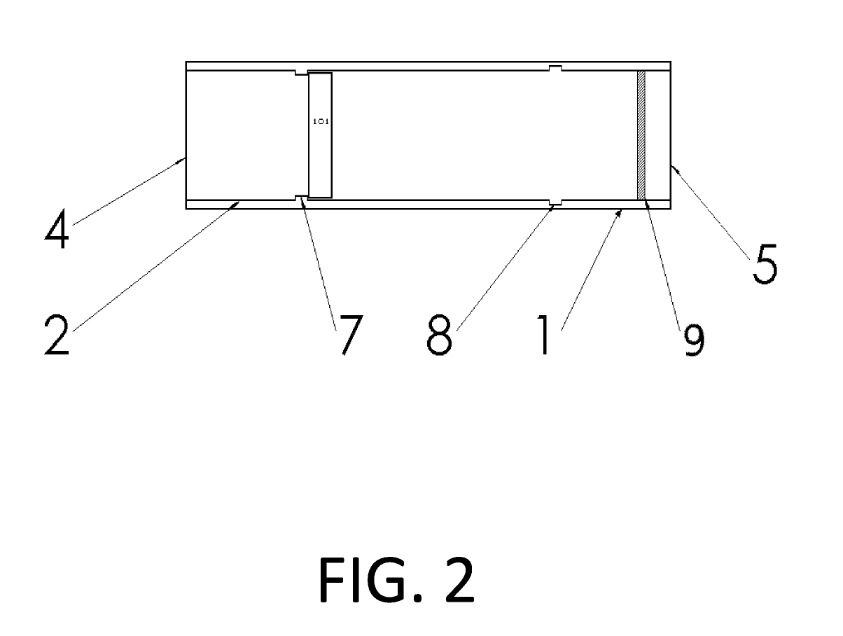

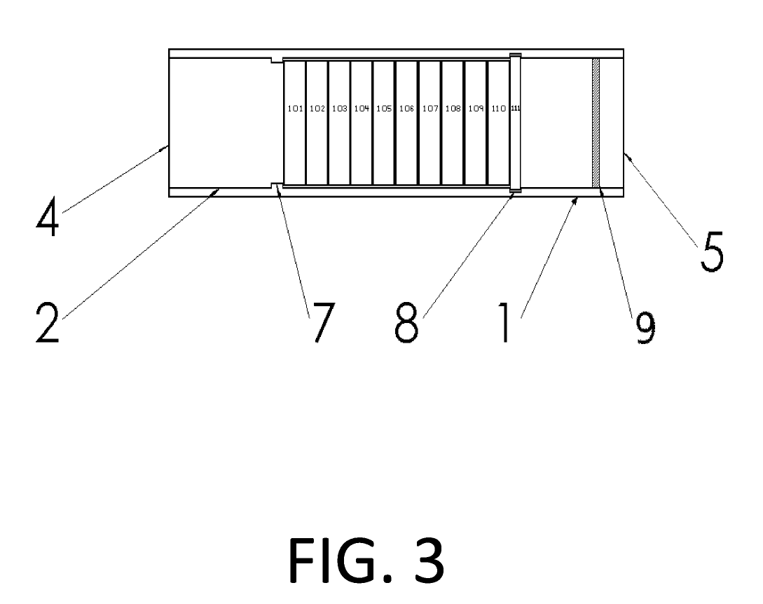

Referring to FIG. 2 and FIG. 3, the present disclosure shows a capillary diagnostic device in accordance with an exemplary embodiment of the present disclosure. In the capillary diagnostic device described herein, it comprises a cylindrical tube (e.g., capillary tube) 1 that includes a proximal end 4 with an opening for contacting a fluid sample. The cylindrical tube 1 also includes a distal end 5 for contacting a cover object that covers the distal end 5, wherein the cover object may include a finger or other pressure equalizing object, or the cover object may include a suction device, and an interior surface 2 with a first inner diameter. The suction device may include a plunger, depressible bulb, or other object configured for causing a negative pressure and/or positive pressure through a channel formed by the interior surface 2 of the cylindrical tube 1. A plurality of rings 101 through 110 that each have a probe selected to test for a one or more genes, antibodies, peptides or other biologic molecules (e.g., analytes) disposed upon the rings, where the plurality of rings are disposed in a sequence and at a distance from the proximal end 4 on the interior surface 2 and the plurality of rings contact the fluid sample. Each probe may be an antibody probe, antigen probe, DNA probe, RNA probe, peptide probe, oligonucleotide probe, or another probe for a biologic identifier. In some embodiments the probe may be located within a biologic material, for example, the probe may be microencapsulated or nanoencapsulated in a chemical substance, such as a polymer or wax, or embedded in a matrix, such as a polymer or wax matrix.

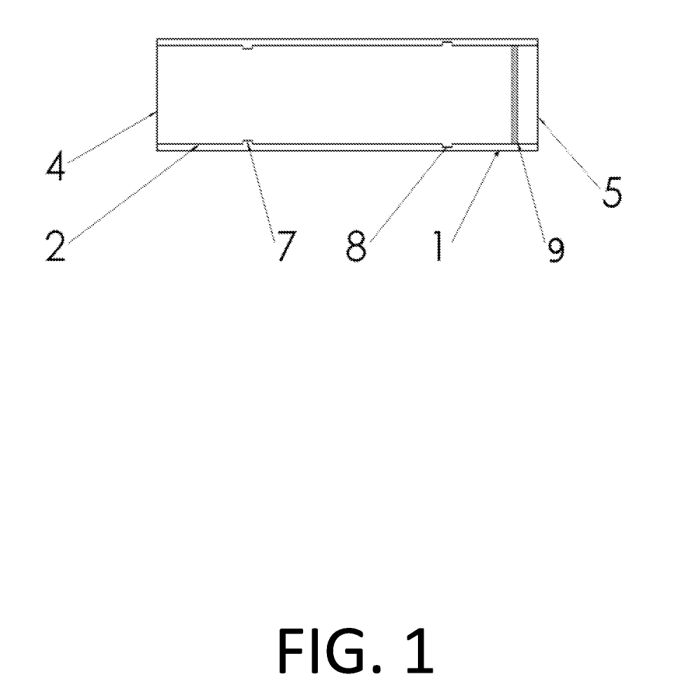

Referring to FIG. 1 and FIG. 2, in some embodiments the capillary diagnostic device may have a concentric lip portion 7 formed by an interior circular ledge as part of the cylindrical tube which a ring may abut, where the ring has a probe disposed thereon. In some embodiments, the concentric lip portion 7 may be formed during the manufacturing process of the capillary tube. The concentric lip portion 7 forms a second inner diameter of the cylindrical tube 1 that is smaller than the first inner diameter of the cylindrical tube 1.

Referring to FIG. 1 and FIG. 2, in further embodiments the capillary diagnostic device will have a circular recess portion 8, that is closer to the distal end 5 of the capillary diagnostic device than the concentric lip portion 7. The circular recess portion 8 has a third inner diameter of the cylindrical tube 1 larger than the first inner diameter of the cylindrical tube 1. The distance between the concentric lip portion 7 and the circular recess portion 8 may depend on the number of rings with probes disposed thereon that will be in the capillary diagnostic device. In some embodiments, the circular recess portion 8 may be created during the manufacturing process of the capillary tube. The circular recess portion 8 may be used to keep the plurality of rings in place. For example, an expandable ring 111 (e.g., an “O” ring) made of a chemically inert and expandable rubber or thermosetting plastic may be placed inside the circular recess portion 8 causing the plurality of rings to be kept in place within the capillary diagnostic device.

Referring to FIG. 1, FIG. 2, and FIG. 3, in some embodiments the capillary diagnostic device may have an indicator 9. The indicator 9 may be configured for demonstrating to a user that the fluid sample has contacted a ring 101 or the plurality of rings 101 through 110, where the indicator 9 is a mark or region on the capillary tube located a distance from the proximal end 4 more distal than the ring 101 in the embodiment of FIG. 2 and the plurality of rings 101 through 110 in FIG. 3. For example, when the fluid sample moves to a position at or distal to the indicator 9, the user is assured the fluid sample has contacted a ring 101 in the embodiment of FIG. 2 and the plurality of rings 101 through 110 in the embodiment of FIG. 3.

In further embodiments a pressure equalizing object or suction device may be connected to the distal end 5 of the cylindrical tube 1 causing a fluid sample to be drawn up the capillary diagnostic device such that the fluid sample flows up the interior surface 2 and covers the ring 101 in the embodiment of FIG. 2 and the plurality of rings 101 through 110 in the embodiment of FIG. 3, where each ring has probes disposed thereon.

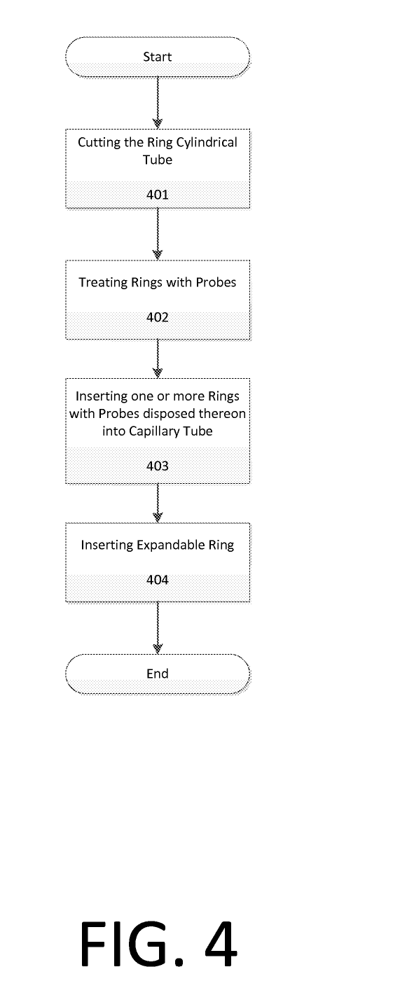

Referring to FIG. 4 described herein is a method for making a capillary diagnostic device. The method for making the capillary diagnostic device described herein begins with the making of one or more rings with a probe disposed thereon. In some embodiments, a ring cylindrical tube is a silane-treated or other coated tube of variable length and has an outer diameter slightly smaller than the first inner diameter of the capillary tube 1, such that the ring cylindrical tube would fit snuggly within the capillary tube 1 but retains the the ability to move in and out of the capillary tube 1 without difficulty. The ring cylindrical tube has a probe disposed on at least that ring cylindrical tube's interior surface. In implementations, to produce a ring, the ring cylindrical tube is cut at variable intervals, such as 1 mm intervals, to produce rings (step 401). In some embodiments, cutting the ring cylindrical tube is done using precision lasers. The one or more rings may be placed in separate dishes each with a different probe and/or necessary organic solvents (e.g., a probe solution) for treatment (step 402). For example, ten probes may be specific short oligonucleotides that are specific to ten genes that are of interest in cancer biology, such as APC, BRCA1, CDK4, HER2, MLH1, MSH2, CMM1, p16, p53, and Rb1. In this example using oligonucleotides, the probes may range from 35 to 100 nucleotides. To continue the example, where the probes are oligonucleotides, each separate dish may have a probe solution, where each probe solution corresponds to a different one of the specific short oligonucleotides and where each probe solution is mixed in TE buffer at 1 micromole (μM) concentration. Depending on the volume of the probe in the probe solution and the volume of the dish, one or more rings (e.g., hundreds or even thousands of rings) can be placed in each separate dish. Further, a ring may be incubated and dried in the process of disposing the probe on the ring.

In another embodiment, one or more rings may be created by first placing the entire ring cylindrical tube in a dish with a probe. Then, the entire ring cylindrical tube may be incubated and dried such that the probe is disposed on at least the interior surface of the ring cylindrical tube. Then the ring cylindrical tube may be cut with, for example, a precision laser at variable intervals thereby creating one or more rings.

Referring to FIG. 2, FIG. 3, and FIG. 4, in some embodiments, after disposal of a probe onto the one or more rings, the one or more rings may be inserted into the capillary diagnostic device (step 403). A ring may be fit inside the capillary tube 1, where the capillary tube 1 is a length, for example 20 mm, and a diameter, for example 5 mm, by first introducing the ring to the distal end 5 the capillary tube 1. Introduction of a ring may be done using tweezers or another grasping tool or process. Next, the ring may be inserted by pushing the ring into the capillary tube 1 using a sterile steel, glass rod, or rod of other composition. In one embodiment, a first ring 101 is inserted into the capillary such that it will abut the concentric lip portion 7. Subsequent rings 102 through 110, each potentially having a different probe disposed thereon, may be inserted via the same mechanism under step 403. After insertion of a last ring 110 into the capillary tube 1, in some embodiments, an expandable ring 111 may be inserted into the capillary tube 1 at step 404. The expandable ring 111 may be inserted into the capillary tube 1 by first introducing the expandable ring 111 to the distal end 5 of the capillary tube 1 using tweezers or another grasping tool or process and further pushing the expandable ring 111 into the capillary tube 1 using a sterile steel, glass, or other composition rod. In this embodiment, the expandable ring 111 is pushed into the capillary tube 1 until the expandable ring 111 reaches the circular recess portion 8, where the expandable ring 111 then fits or snaps into the circular recess portion 8. In some embodiments, the expandable ring 111 may be an “O” ring, which once fit into the circular recess portion 8 will remain in place and hold in place the one or more rings with probes disposed thereon. In other embodiments, the expandable ring 111 may be made of thermosetting plastic, so that after the expandable ring 111 is fit or snapped into the circular recess portion 8, the expandable ring 111 may be heat treated, causing the expandable ring 111 to remain in place. With the expandable ring 111 remaining in place, the one or more rings with probes disposed thereon also remain in place.

In other implementations, the cylindrical tube 1 may not have the circular recess portion 8 or the concentric lip portion 7. Instead, one or more rings with probes disposed thereon may be secured by other means. For instance, a ring may be secured to the interior surface 2 of the cylindrical tube 1 by a process of annealing the ring to the interior surface 2 (e.g., by heating or by adhesive coating on the ring's outer surface). In other embodiments, a ring may be secured to the interior surface 2 of the cylindrical tube 1 by a process of threading, wherein the ring's outer surface is threaded to engage a threaded interior surface of the cylindrical tube. In other embodiments, a ring may be secured to the interior surface 2 of the cylindrical tube 1 by using a rubber stopper ring and an hour glass formation or shaping on the interior surface of the cylindrical tube 1 set a distance from the proximal end 4. In such an embodiment, the hour glass formation or shaping is a strategically narrowed or contoured area of at least the interior surface 2 of the cylindrical tube 1. Further, the hour glass formation or shaping acts as a first stopper for the first ring inserted into the cylindrical tube 1 and the rubber stopper acts as a second stopper following insertion of the first ring if only one ring is inserted into the cylindrical tube 1 or the last ring if two or more rings has been inserted into the cylindrical tube 1.

In some embodiments, the distance of the one or more rings 101 through 110 from the proximal end 4 depends on the diameter of the capillary tube 1. In some embodiments, once all of the rings are set in place, the capillary diagnostic device may be used. In some embodiments, once all of the rings are set in place, the capillary diagnostic device may be used in combination with a cover object (e.g., a finger). In some embodiments, once the one or more rings are set in place, the capillary diagnostic device may be used once combined with a pressure equalizing object appended to the capillary diagnostic device. In yet another embodiment, once the one or more rings are set in place, the capillary diagnostic device may be used once combined with a suction device appended to the capillary diagnostic device. In some embodiments, the capillary diagnostic device may be a single use device that may be disposed of after a fluid sample is analyzed.

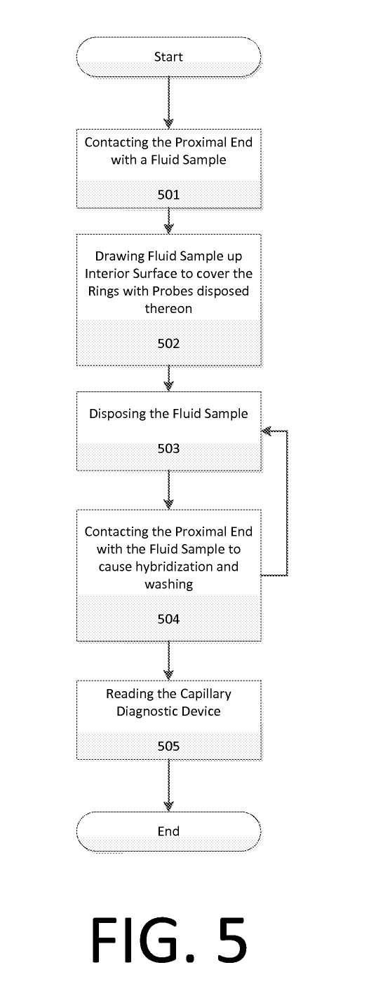

Referring to FIG. 5, a method for use of the capillary diagnostic device to analyze a fluid sample is disclosed herein. In some embodiments, the fluid sample may be a biologic sample that is being analyzed, for instance blood, plasma, protein extracts from cells (plant or animal), DNA or RNA from any source, semen, urine, or cell suspensions. In some embodiments, the fluid sample to be analyzed may be human genomic DNA, which is labeled via nick translation, direct labeling PCR, or random priming. In other embodiments of the present disclosure, a fluid sample containing human genomic DNA from multiple subjects may be prepared by labeling each subject's fluid sample with a specific dye and then combining the separate fluid samples for testing via the capillary diagnostic device. In yet another embodiment, the fluid sample may be reagents that are used for subsequent steps in the process, for example, buffers, ethanol, isopropanol, water or other organic solvents.

Referring to FIG. 5, in step 501, the capillary diagnostic device contacts the proximal end 4 of the capillary diagnostic device with the fluid sample to be analyzed. Next, in step 502, the capillary diagnostic device draws the fluid sample up the capillary tube 1. In some embodiments, depending on the diameter of the capillary tube 1 and/or the volume of the fluid sample, the fluid sample will draw up the capillary tube 1 by capillary action (e.g., capillary action method). For example, if the cylindrical tube 1 has a diameter of 2 mm, the capillary action method can cause the fluid sample to contact the one or more rings of the capillary diagnostic device with probes disposed thereon. In another embodiment, for a capillary tube 1 of greater diameter, for example 5 mm, the distal end 5 of the capillary diagnostic device may be covered with a cover object and then inverted (or tilted), drawing the fluid sample up the interior surface 2 of the capillary tube 1 to contact the one or more rings with the probes disposed thereon (e.g., pressure equalizing object method). In another embodiment, for a cylindrical tube of greater diameter, for example 5 mm, a suction device may be appended to the distal end 5 of the capillary diagnostic device for drawing the fluid sample up the interior surface 2 of the capillary diagnostic device to contact the one or more rings with probes disposed thereon (e.g., suction device method). Under the pressure equalizing object method, the fluid sample will remain in the capillary diagnostic device so long as a pressure equalizing object remains applied to the distal end 5 of the capillary diagnostic device. Under the suction device method, the fluid sample will remain in the capillary diagnostic device so long as a the suction device remains engaged at the distal end 5 of the capillary diagnostic device. For any of the capillary action, pressure equalizing object, or suction device methods, the capillary diagnostic device may be moved to a disposal container to allow the fluid sample to flow out of the capillary diagnostic device into the disposal container (step 503).

In some embodiments, the analyte being tested for may require contacting the one or more rings having probes disposed thereon with one or more buffers to carry out hybridization and washing steps necessary to detect the presence of the analyte (step 504). For example, the analyte of interest may be an antibody present in human plasma, so hybridization and washing steps done in a ELISA type assay may be used. After the disposal of the fluid sample to be analyzed, the following fluids may be introduced to the capillary diagnostic device in the following order: a buffer to wash the one or more probes at least one time, a buffer causing a blocking agent (e.g., non-specific proteins) to cover unbound sites, and a buffer to wash the one or more probes at least one time. In some embodiments, a buffer may contain a secondary antibody that is configured to detect the bound antibodies, such that the secondary antibody will bind to all analytes. Attached to the secondary antibody will be a fluorescent dye causing detection of an analyte upon reading the capillary diagnostic device. The introduction and disposal of subsequent fluids may be carried out using the capillary action method, pressure equalizing object method, or the suction device method.

Referring to FIG. 5, after the completion of the steps to detect the analyte, the capillary diagnostic device may be read (step 505). For example, reading the capillary diagnostic device may mean analyzing the one or more probes for fluorescence, chemiluminiscence, absorbance, electrical changes, or other type of biologic assay indicators to demonstrate the binding of an analyte to the one or more probes.

What is claimed:

1. A capillary diagnostic device for analyzing a fluid sample from a subject, the capillary diagnostic device comprising:
a capillary tube including a proximal end with an opening for receiving the fluid sample, a distal end generally opposite the proximal end with an opening for contacting a cover object, and an interior surface; and
a ring, the ring being seated within the capillary tube, the ring being spaced at a distance from the proximal end on the interior surface, the ring being configured to contact the fluid sample, and the ring having a probe disposed thereon, the probe being selected for testing for the presence of an analyte in the fluid sample.

2. The device of claim 1 wherein:
the interior surface includes a first inner diameter, a concentric lip portion with a second inner diameter that is shorter than the first inner diameter, and a concentric recess portion with a third inner diameter that is longer than the first inner diameter.

3. The device of claim 2 further comprising:
an expandable ring that fits into the concentric recess portion and abuts the ring, which abuts the concentric lip portion.

4. The device of claim 1 further comprising:
a plurality of additional rings, each additional ring being seated within the capillary tube, a first additional ring abuts the ring, each additional ring seated in sequential order at increasing distances from the proximal end on the interior surface, each additional ring being configured to contact the fluid sample, and each additional ring having a probe disposed thereon, each probe being selected for testing for the presence of one or more analytes in the fluid sample.

5. The device of claim 1 wherein:
the cover object is a pressure equalizing object configured for equalizing air pressure within the capillary tube.

6. The device of claim 1 wherein:
the cover object is a suction device configured for creating negative air pressure in the capillary tube.

7. The device of claim 1 wherein:
the probe is selected from a group consisting of antibodies, antigens, DNA, RNA, peptides, and oligonucleotides.

8. The device of claim 1 wherein:
the probe is selected from a group consisting of microencapsulated probes, nanoencapsulated probes, and matrix embedded probes.

9. The device of claim 1 wherein the capillary tube includes an indicator.

10. A method of making a capillary diagnostic device for analyzing a fluid sample from a subject, the method comprising:
cutting a cylindrical ring tube at intervals to produce a ring;
treating the ring with a probe solution to dispose of a probe on the ring, the probe being selected for testing for the presence of an analyte in the fluid sample; and
setting the ring, having the probe disposed thereon, onto an interior surface of a capillary tube, the capillary tube having a proximal end and a distal end generally opposite the proximal end.

11. The method of claim 10 further comprising:
setting a plurality of additional rings, each additional ring having a probe disposed thereon, onto the interior surface of the capillary tube, a first additional ring of the plurality of additional rings abutting the ring on the interior surface of the capillary tube.

12. The method of claim 10 further comprising:
setting an expandable ring into a concentric recess portion of the interior surface, where the interior surface has a first inner diameter, a second inner diameter defined by a concentric lip portion that is shorter than the first inner diameter, and a third inner diameter defined by the concentric recess portion that is longer than the first inner diameter.

13. The method of claim 10 wherein cutting is performed by a laser.

14. The method of claim 10 wherein the probe is selected from the group consisting of antibodies, antigens, DNA, RNA, peptides, and oligonucleotides.

15. A method of using a capillary diagnostic device for analyzing a fluid sample from a subject, the method comprising:
contacting the capillary diagnostic device with the fluid sample, the capillary diagnostic device having a proximal end with an opening for receiving the fluid sample, a distal end generally opposite the proximal end with an opening for contacting a cover object, and an interior surface, the interior surface having a ring with a probe disposed thereon, the ring being spaced at a distance from the proximal end, the proximal end contacting the fluid sample;
drawing the fluid sample from the proximal end toward the distal end of the interior surface to cause the fluid sample to contact the ring;
releasing the fluid sample from the capillary diagnostic device; and
reading the probe disposed on the ring.

16. The method of claim 15, wherein releasing the fluid sample from the capillary diagnostic device includes removing the cover object from the distal end to expel the fluid sample.

17. The method of claim 16, before reading the probe disposed on the ring, further comprising:
drawing a second fluid for washing the probe from the proximal end toward the distal end of the interior surface to cause the second fluid to contact the ring;
releasing the second fluid from the capillary diagnostic device;
drawing a third fluid for hybridizing the probe; and
releasing the third fluid from the capillary diagnostic device.

18. The method of claim 17 wherein releasing the second fluid and releasing the third fluid from the capillary diagnostic device includes removing the cover object from the distal end to expel each of the second fluid and the third fluid.

19. The method of claim 15 wherein the probe is selected from the group consisting of antibodies, antigens, DNA, RNA, peptides, and oligonucleotides.

20. The method of claim 15 wherein the fluid is drawn until the fluid reaches an indicator on the capillary diagnostic device.
